(19) United States
(12) Patent Application Publication
(10) Pub. No.: US 2010/0203624 A1
(43) Pub. Date: Aug. 12, 2010

(54) THREE DIMENSIONAL DISPOSABLE BIOREACTOR

(75) Inventor: Vijay Singh, Bernardsville, NJ (US)

Correspondence Address: GE HEALTHCARE BIO-SCIENCES CORP.;PATENT DEPARTMENT, 101 CARNEGIE CENTER, PRINCETON, NJ, 08540, US

(73) Assignee: GE HEALTHCARE BIOSCIENCE BIOPROCESS CORP., PISCATAWAY, NJ (US)

(21) Appl. No.: 12/676,180

(22) PCT Filed: Sep. 15, 2008
(86) PCT No.: PCT/US08/76372

§ 371 Date: Mar. 3, 2010

Related U.S. Application Data
(60) Provisional application No. 60/975,213, filed on Sep. 26, 2007.

(51) Int. Cl. C12M 1/02

(52) U.S. Cl. 435/289.1

(57) ABSTRACT

A bioreactor including a support, a container, means to secure the container on the support and nutrients and cell in the container. The container has top and bottom walls joined to form a chamber having a portion of the top and bottom walls joined by side walls; and end walls connected to the top and bottom walls forming a three dimensional container. The support is pivotally mounted to a base and driven about a single axis and the end walls of the container are transverse to the single axis.

### CROSS-REFERENCE TO RELATED APPLICATIONS

This application is a filing under 35 U.S.C. §371 and claims priority to international patent application number PCT/US2008/076372 filed Sep. 15, 2008, published on Apr. 2, 2009, as WO 2009/042432, which claims priority to U.S. provisional patent application No. 60/975,213 filed on Sep. 26, 2007; the disclosure of which is incorporated herein by reference in its entirety.

### FIELD OF THE INVENTION

The present disclosure relates to hermetically sealed bags containing products used in the pharmaceutical and biotechnology processing industries and, more particularly, to disposable cell bags or bioreactors.

### BACKGROUND OF THE INVENTION

The bio-processing industry has traditionally used stainless steel systems and piping in manufacturing processes for fermentation and cell culture. These devices are designed to be steam sterilized and reused. Cleaning and sterilization are however costly labor-intensive operations. Moreover, the installed cost of these traditional systems with the requisite piping and utilities is often prohibitive. Furthermore, these systems are typically designed for a specific process, and cannot be easily reconfigured for new applications. These limitations have led to adoption of a new approach over the last ten years—that of using plastic, single-use disposable bags and tubing, to replace the usual stainless steel tanks.

In particular bioreactors, traditionally made of stainless-steel, have been replaced in many applications by disposable bags which are rocked to provide the necessary aeration and mixing necessary for cell culture. These single-use bags are typically provided sterile and eliminate the costly and time-consuming steps of cleaning and sterilization. The bags are designed to maintain a sterile environment during operation thereby minimizing the risk of contamination.

Commonly used bags are of the “pillow style,” mainly because these can be manufactured at low cost by seaming together two flexible sheets of plastic.

One of the successful disposable bioreactor systems uses a rocking table on to which a bioreactor bag is placed. The bioreactor bag is partially filled with liquid nutrient media and the desired cells. The table rocks the bag providing constant movement of the cells in the bag and also aeration from the turbulent air-liquid surface. The bag, typically, has a gas supply tube for the introduction of air or oxygen, and an exhaust gas tube to allow for the removal of respired gases. Nutrients can be added through other tubes.

One possible limitation of this type of device is that it may be difficult to scale up beyond a few hundred liters because poor liquid circulation causes nutrient and waste gradients that inhibit cell performance. This is because the back and forth motion of the single-axis rocker used in these applications creates good liquid circulation in the direction perpendicular to the rocking axis, but relatively little mixing in the direction parallel to the rocking axis. In large volume bags (greater than 100 liters), or in bags with a large length to width ratio, this poor axial circulation can result in a long time to achieve homogeneity of the bag contents. This makes pH control in the bioreactor bag difficult, since additions of acid or base added to the bioreactor to modulate the pH can take a long time to disperse throughout the bag. Nutrients added to the bioreactor bag may not be distributed uniformly. Poor liquid circulation also limits the amount of oxygen that can be transferred from the head space, and thus the maximum concentration of cells that can not be cultured.

Circulation flow can be improved by incorporating a second axis of rotation. By synchronizing the two axes it is possible to impart a gyratory motion that greatly improves mixing and mass transfer. However, the addition of second axis increases the cost tremendously, and the increase in mechanical complexity makes the rocker less reliable and more difficult to maintain.

Therefore, there is a need for an apparatus that enables a user to scale up the mixing of nutrient media in a bioreactor bag. Also, there is need for a bioreactor bag that makes it simple to control the pH where the addition of acid or base to the bioreactor bag does not take a long time. Further, there is need for a bioreactor bag where the amount of oxygen is not limited so the maximum concentration of cultures can be cultured.

### SUMMARY OF THE INVENTION

The present invention has been accomplished in view of the above-mentioned technical background, and it is an object of the present invention to provide a bioreactor bag that enables a user to scale up the mixing of nutrient media and efficiently mix the nutrient media in the bioreactor bag.

A bioreactor including a support, a container, means to secure the container on the support and nutrients and cell in the container. The container has top and bottom walls joined to form a chamber having a portion of the top and bottom walls joined by side walls; and end walls connected to the top and bottom walls forming a three dimensional container. The support is pivotally mounted to a base and driven about a single axis and the end walls of the container are transverse to the single axis.

The container walls are flexible sheets seamed together. The top and bottom walls are seamed together and portions of the top and bottom wall form the side walls of the chamber. The end walls, which are transverse to the single axis, are panels seamed to the top, bottom and side walls. Alternatively, The container may be a modeled structure.

The juncture of the side and end walls are non-orthogonal so as to induce a swirling motion of liquids in the chamber when the container is rocked about the single axis. A first pair of opposed junctures are obtuse angles and a second pair of opposed junctures are acute angles. The junctures may be arcuate. The junctures may include a linear wall of the chamber connecting and being oblique to both a side and an end wall. A first pair of opposed junctures may have a first length and a second pair of opposed junctures have a second length shorter than the first length so as to produce a single direction of swirling during rocking.

Baffles may be connected to the top and bottom walls and displaced from the side and end walls to induce a swirling motion of liquids in the chamber when the container is rocked about the single axis.

### BRIEF DESCRIPTION OF DRAWINGS

These and other advantages of the present invention will become more apparent as the following description is read in conjunction with the accompanying drawings, wherein:

FIG. 1 shows a perspective view of a pillow style two dimensional bag construction according to the prior art.

FIG. 2 shows a perspective view of the bag of FIG. 1 secured to rocking bioreactor based on prior art.

FIG. 3 shows the liquid flow pattern of the bag of FIG. 1 resulting from a single axis of rocking based on prior art.

FIG. 4 shows a perspective of a three-dimensional bag construction according to another embodiment of the invention.

FIG. 5 shows a perspective view of the embodiment shown in FIG. 4 depicting details of construction according to an embodiment of the invention.

FIG. 6 shows a baffled bag and the single liquid flow pattern resulting from a single axis of rocking according to a first embodiment of the invention.

FIG. 7 shows a baffled bag and the single liquid flow pattern resulting from a single axis of rocking according to a second embodiment of the invention.

FIG. 8 shows a baffled bag and the dual liquid flow pattern resulting from a single axis of rocking according to a third embodiment of the invention.

FIG. 9 shows a baffled bag with linear baffles and the liquid flow pattern resulting from a single axis of rocking according to a fourth embodiment of the invention.

FIG. 10 shows a trapezoidal shaped baffled bag and the liquid flow pattern resulting from a single axis of rocking according to a fifth embodiment of the invention.

FIG. 11 shows another embodiment with internal flow diversion baffles.

### DETAILED DESCRIPTION OF THE PREFERRED EMBODIMENTS

The presently preferred embodiments of the invention are described with reference to the drawings, where like components are identified with the same numerals. The descriptions of the preferred embodiments are exemplary and are not intended to limit the scope of the invention.

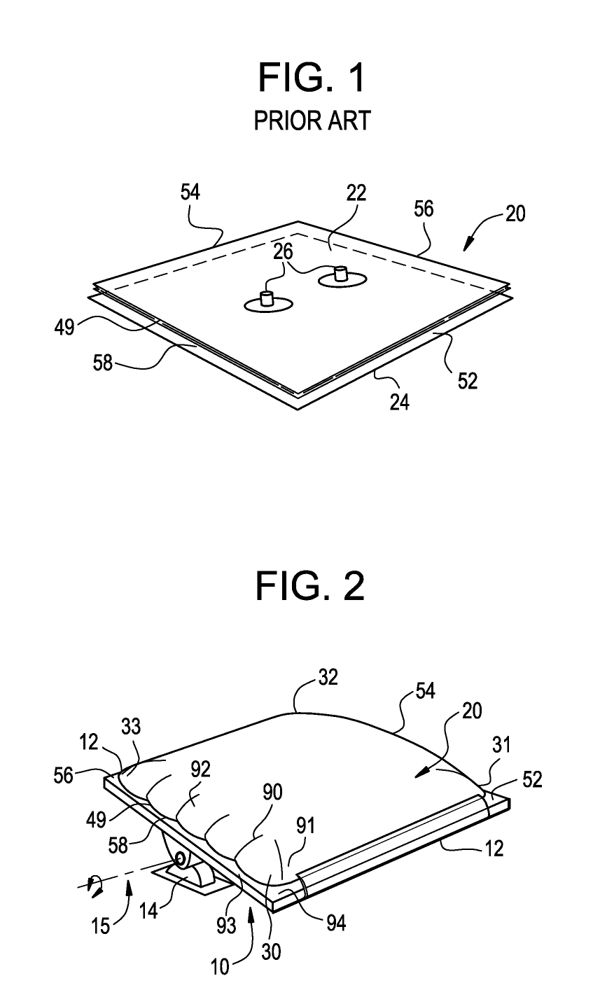

A prior art bag 20, as shown in FIG. 1, is a flat, rectangular, “pillow-style” cell culture bag 20 commonly used in rocking bioreactor applications, for example in the system of U.S. Pat. No. 6,190,913 entitled “Method for Culturing Cells Using Wave-Induced Agitation” filed Aug. 12, 1998, which is hereby incorporated by reference. The bag 20 is formed by seaming together top sheet 22 to bottom sheet 24. The outline seam 49 formed by sealing the two sheets together at all four edges 52, 54, 56, and 58 which bounds the inside chamber in which the culture fluids 32 are contained. Ports 26 on top sheet 22 are used for the introduction and exhaust of gases.

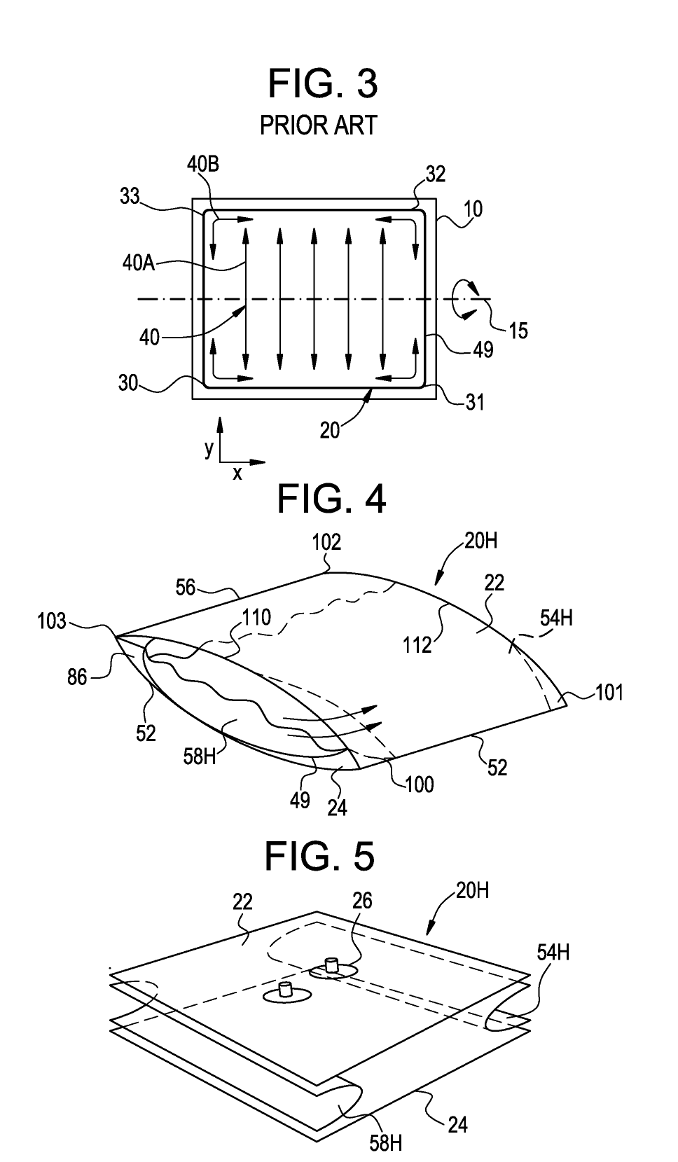

FIG. 2 shows the bag 20 secured to a support 10 of a rocking bioreactor using clamps 12 on side edges 52 and 54. The support 10 is pivotable mounted to a base 14 and is rocked about a single axis 15. The fluid flow induced by the rocking is depicted in FIG. 3 as streamlines 40. As shown, the rocking motion generates fluid motion 40A mainly along the Y-axis (perpendicular to rocking axis 15). Very little fluid motion 40B in the X-direction (parallel to rocking axis 15) is generated in the substantially orthogonal corners 30-33. It can take considerable time to mix the contents of mixing bag 20 to homogeneity in the structure of bag 20. The mixing time can be reduced by increasing the rocking speed, but this puts more stress on the bag leading to possible breakage and also increases the energy requirements for mixing.

As shown in FIG. 2 for the two dimensional bag 20, creases and wrinkles 90, 91, 92 may form on upper surface 22 of each corner 30-33, and also 93, 94 on the underside 24 of each corner 30-33 of the bag 20. Excess material may develop in corners 30-33 because the inflation pulls in unrestrained end edges 56 and 58, and pushes out corners 30-33. This excess material cannot be inflated to rigidity, and may flop around during rocking, which could lead to premature fatigue failure. The bag 20 is stressed when inflated and this stress is transmitted through top sheet 22 and bottom sheet 24 to clamped edges 52 and 56. The stress is distributed along edges 52 and 56, but not at corners 30-33 which are not pulled taut into support 10 due to the excess material present at corners 30-33. Consequently, failures can occur at corners 30-33 where the bag cannot be maintained taut and rigid.

FIG. 4 depicts a three-dimensional cell culture bag 20H, or container or bioreactor formed by forming the end walls 54H and 58H as gussets on side walls 52 and 56. Culture bag 20H is formed from a multiplicity of flat flexible panels 22, 24, 54H, and 58H as depicted the exploded view shown in FIG. 5. This figure shows one way in which culture bag 20H can be formed by seaming together two flexible sheets 22 and 24, folding in two smaller panels 54H and 58H, and closing them off by a cross or curved seam 49 which bounds the inner volume of culture bag 20H. The segments of the seam 49 which joins the gusseted end walls 54H and 58H to the top sheet 22 and the bottom sheet 24 is arcuate so as to form the baffles in FIG. 4. The segments of the seam 49 which joins the top sheet 22 and the bottom sheet 24 forms the side walls 52 and 56.

When culture bags 20H are restrained at edges 52 and 56, and inflated, top sheets 22 and bottom sheet 24 are able to separate at the gusseted end walls 54H and 58H. The culture bag 20H conforms to the inflated three-dimension shape without wrinkles, creases, or excess corner material. Corners 100-103 are now pulled taut and provide additional structural elements that distribute stress from the high points 110 and 112 of culture bag 20H to the clamped edges 52 and 56. These edges are clamped along their entire length to holder 10, and form anchor points to restrain the bag from over inflating. The corner sections 100-103 also function as a reinforcing structure to support the bag during rocking. Rocking towards edge 56 about axis 15 causes culture bag 20H to be pulled up from edge 52. This movement is resisted by corners 100 and 101 that serve to hold culture bag 20H down. The additional tendency for the bag to slide towards edge 56 is resisted by corners 100 and 101. In the reverse stroke the same functionality is provided by corners 102 and 103.

The improved bags 20 can be a molded three-dimensional structure or fabricated by seaming flexible sheets. The edges and gusset may be curved seams, or manufactured as a series of straight line seam segments as shown herein.

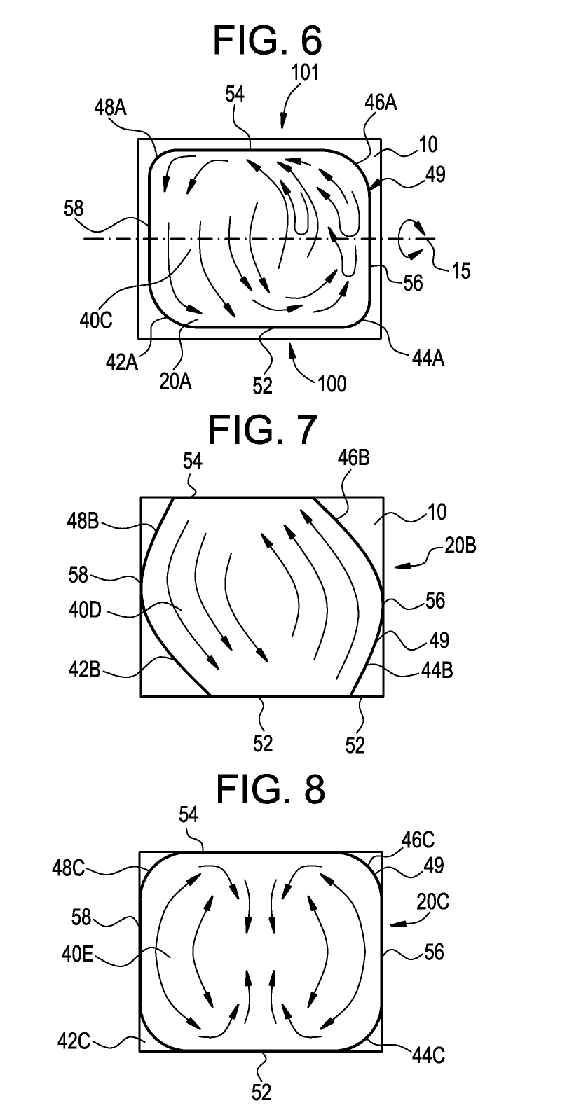

Referring first to the embodiment shown in FIG. 6 a cell culture bag 20A bounded by seams 49, containing components, at least one of which is a liquid, to be mixed. Cell culture bag 20A is placed on a rocking platform 10, which pivots about rocking axis 15. Corner 42A of the cell culture bag 20A, is contoured in the same manner as the diagonally opposite corner 46A, an arc that forms these corners can have a radius ranging from ¼ to ½ the width of the cell culture bag 20A but is different from adjacent corner 44A which is contoured in the same manner as its diagonally opposite corner 48A with another arc radius ranging from 1/20 to ¼ the width of the cell culture bag 20A. The corners 42A and 46A are baffles or flow directors formed at the juncture of the side walls 52, 54 and the end walls 56, 58. These baffles or flow directors force the liquid in the bag to follow the contour as the liquid cannot pass through liquid-tight seam 49 formed by joining top sheet 22 to bottom sheet 24. The corners form a juncture of the side and end walls, which is transverse to the single axis of rocking 15 to induce a circulatory swirling motion of the liquid in the chamber when the cell culture bag is rocked. The liquid may consist of soluble powders in liquid, low or high viscous liquids that are designed to be mixed or blended together. In bag 20A, the junctures are oblique and arcuate. The oblique junctures have obtuse angles of 90 degrees plus while the arcuate junctures have angles of less than 90 degrees.

The effect of this asymmetry of cell culture bag 20A is that as it is tipped towards 100 by tilting support 10, liquid flows due to gravity from edge 54 towards edge 52. As the liquid approaches edge 52 it is diverted right towards the center of the cell culture bag 20A by corner 42A. Liquid on the opposite side flows into corner 44A which not shaped so as to divert flow to the center. This imbalance of flow velocities forces flow from the left end 58 to the right end 56, and flow from the right end is inhibited from entering the left end. On the reverse stroke, support 10 tilts towards 101, and the liquid in cell culture bag 20A flows from edge 52 towards edge 54. Liquid entering corner 46A is diverted to the center of container 20A due to the shape of corner 46A, while liquid entering corner 48A is not diverted towards the center. After 2 to 5 rocking strokes a self sustaining motion develops, and is sustained as long as the bag is rocked, as shown by the fluid streamlines 40C with the liquid in the bag circulating counter-clockwise. This self-sustaining motion persists as long as the rocking motion is continued. This circulatory motion is superposed on the back and forth motion and is very effective at mixing fluid parallel to the rocking axis 15, a major limitation with prior art. The circulatory motion can easily be reversed to the clockwise direction by interchanging the geometry of the corners.

FIG. 7 shows a cell culture bag 20B which produces a greater circulation than in cell culture bag 20A. This is because the radius of the corners 42B and 46B is larger that their counterparts 42A and 46A (FIG. 6). The radius of these corners can range from ½ to 2× the width of the mixing bag 20B. Corners 44B and 48B also have a larger radius (¼ to ½ the mixing bag width) than their counterparts 44A and 48A (FIG. 6). These larger radius arcs provide a more gentle flow pattern, reducing some of the turbulence caused by the sharp corners 44A and 48A. The resulting circulation is shown as 40D. It is critical that asymmetry of the adjacent corners be maintained. For example, the symmetrical cell culture bag 20C shown in FIG. 8 has small equal arcuate corners 42C through 48C. The resulting circulation 40E has very little fluid circulation parallel to the rocking axis 15 and is therefore similar to prior art cell culture bags with relatively poor mixing.

The flow contours can be molded in the bag as curved surfaces or fabricated by seaming sections of plastic. The contours may be curved seams, or manufactured as a series of straight line seam segments. The seams are made by welding together the top and bottom sheet. Various methods—heat sealing, ultrasonic etc are commonly used. Straight seams can be easily made by inexpensive thermal bar sealers. Curved seams are much more difficult and are typically made using heated platens. These are expensive and designed for specific bag sizes. The laser method has the advantage that any shape seam, bag geometry or size can be made by just changing the software.

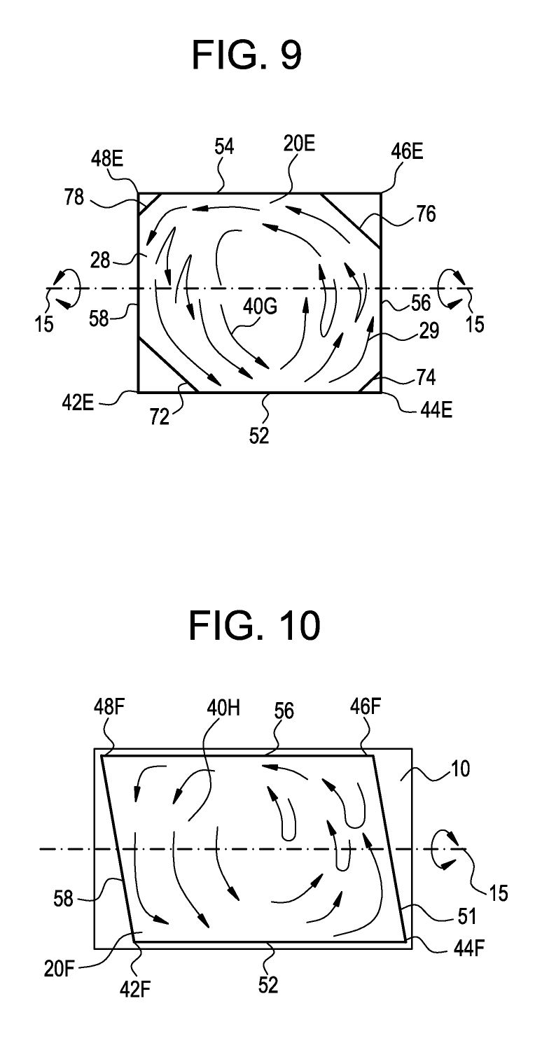

Manufacturing curved seams in bags is difficult, and requires complex equipment. Straight line seams can be easily made using commercial bar type heat sealers. The embodiment shown in FIG. 9 illustrates a cell culture bag made using straight or linear seams that achieves circulation flow. The outer seam 49 forms portions 52, 54, 56, 58 of the inner chamber of the mixing bag 20E that contains the liquid and components to be mixed. Seam 51 defines the baffles 72, 74, 76, 78 as linear segments at the corners and are connected to the top, bottom, side and ends walls. These baffles are typically oriented at 45 degrees (angles from 30 to 60 degrees can be used) to the rocking axis 15. The longer baffles 42E and 46E can extend from ¼ to ⅓ of the length of the side of bag 20E and the shorter baffles 44E and 48E are typically ⅕ to ½ the length of the longer baffles. The resulting circulation of the asymmetrical baffles is shown as 40G. The corners may be removed where they extend beyond the baffles. Also the seam 49 need not extend past the juncture of the baffles, the inner seam 51, to the side and end walls.

The embodiment shown in FIG. 10 illustrates a cell culture bag made using straight or linear seams that achieves circulation flow by changing the shape of cell culture bag 20F from an essentially rectangular form into a trapezoidal shape. The end walls 54 and 58 of the cell culture bag 20F are parallel to each other, but are not perpendicular to the side walls 52 and 56. This creates a first pair of opposed junctures and baffles 42F and 46F that are obtuse angles ranging from 100 to 130 degrees and a second pair of opposed junctures and baffles 44F and 48F that are corresponding acute angles. By setting the obtuse and acute angles automatically fixes the shapes as a parallelogram. When the cell culture bag 20F is rocked about axis 15, the fluid circulates in the direction shown by the flow streamlines 40H effectively mixing the contents of cell culture bag 20F.

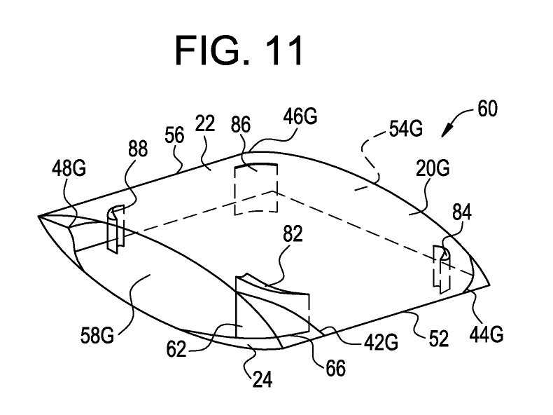

With a two and three dimensional bags, the baffles formed by the intersection of the side and end walls may not have sufficient height when the bag is inflated for the liquid level and rocking motion to produce the desired amount of circulation. The embodiment shown in FIG. 11 illustrates a cell culture bag 20G wherein the baffles are separated and displaced from the intersection of the side and end walls. The baffles 82, 84, 86, 88 are adjacent to the corners 42G, 44G, 46G, 48G. The baffles are connected to the top wall 22, the bottom wall 24, the side walls 52, 56 and the end walls 54G, 58G. The baffles may be connected to the top and bottom walls first and then joined to the side and ends walls when they are formed or joined to the top and bottom walls.

This invention provides an apparatus that enables a user to scale up the mixing of nutrient media in a bioreactor bag. This apparatus makes it simple to control the ph where the addition of acid or base to the bioreactor bag does not take a long time to obtain. Thus, this invention provides the user with a simple method to scale up the mixing of nutrient media in a bioreactor bag.

Although the present bag has been described and illustrated in detail, it is to be clearly understood that this is done by way of illustration and example only and is not to be taken by way of limitation. The scope is to be limited only by the terms of the appended claims.

What is claimed is:

1. A bioreactor comprising;
a support configured to retain a container, wherein the container includes a chamber with liquids;
means to secure the container on the support;
the container includes:
a top wall and a bottom wall joined to form a chamber having a portion of the top and bottom walls joined by a plurality of side walls;
a plurality of end walls connected to the top wall and the bottom wall forming a three dimensional container; and
a plurality of baffles connected to the top wall and the bottom wall at a juncture of the plurality of side walls and the plurality of end walls being transverse to the movable axis, wherein the plurality of baffles are configured to induce a swirling motion of the liquid in the chamber when the container is moved along the movable axis.

2. The bioreactor of claim 1, wherein the support is pivotally mounted to a base about the movable axis.

3. The bioreactor of claim 1, wherein the top wall, the bottom wall, the plurality of side walls and the plurality of end walls are flexible sheets seamed together.

4. The bioreactor of claim 2, wherein the top wall and the bottom wall are seamed together and portions of the top wall and the bottom wall form the plurality of side walls of the chamber and the plurality of end walls that are transverse to the single axis, are panels seamed to the top wall, the bottom wall and the plurality of side walls.

5. The bioreactor of claim 1, wherein the juncture of the plurality of side walls and the plurality of end walls are non orthogonal that induces the swirling motion of liquids in the chamber when the container is rocked about the movable axis.

6. The bioreactor of claim 5, wherein the plurality of baffles have a first pair of opposed junctures that are obtuse angles and the plurality of baffles have a second pair of opposed junctures that are acute angles.

7. The bioreactor of claim 6, wherein the first pair of opposed junctures have a first length and the second pair of opposed junctures have a second length shorter than the first length so as to produce a single direction of swirling during rocking
